(19) United States
(12) Patent Application Publication
(10) Pub. No.: US 2026/0132377 A1
(43) Pub. Date: May 14, 2026

(54) HEMATOPOIETIC STEM CELL PROLIFERATION MEDIUM AND USE OF THE SAME

(71) Applicant: Food Industry Research and Development Institute, Hsinchu (TW)

(72) Inventors: Ming-Wen Su, Hsinchu (TW); Cheng-Hao Wen, Hsinchu (TW); Yu-Jen Chang, Hsinchu (TW)

(73) Assignee: Food Industry Research and Development Institute, Hsinchu (TW)

(21) Appl. No.: 19/219,705

(22) Filed: May 27, 2025

(30) Foreign Application Priority Data
Nov. 8, 2024 (TW) 113142972

(51) Int. Cl. C12N 5/0789

(52) U.S. Cl. CPC C12N 5/0647; C12N 2500/25; C12N 2500/46; C12N 2500/84; C12N 2501/125; C12N 2501/145; C12N 2501/2303; C12N 2501/2306; C12N 2501/26

(57) ABSTRACT

A hematopoietic stem cell (HSC) proliferation medium includes a basal medium, 0.1 ng/mL to 200.0 ng/mL of thrombopoietin (TPO), 0.1 ng/mL to 200.0 ng/mL of stem cell factor (SCF), 0.1 ng/mL to 20.0 ng/mL of interleukin-3 (IL-3), 0.1 ng/mL to 100.0 ng/mL of interleukin-6 (IL-6), 0.1 ng/mL to 200.0 ng/mL of Flt-3 ligand (FL), 0.1 μg/mL to 20.0 μg/mL of insulin, 0.1 μg/mL to 120.0 μg/mL of transferrin, 0.1 g/L to 3.0 g/L of human serum albumin (HSA), 0.1 μM to 4.0 μM of pyrimido-indole derivative UM729, and 0.1 μM to 2.0 μM of aryl hydrocarbon receptor (AhR) antagonist StemRegenin 1 (SR1). Methods for in vitro proliferation of hematopoietic stem cells (HSCs) and in vitro production of hematopoietic progenitor cells (HPCs) using the HSC proliferation medium are also provided.

### CROSS-REFERENCE TO RELATED APPLICATION

This application claims priority to Taiwanese Invention Patent Application No. 113142972, filed on Nov. 8, 2024, the entire disclosure of which is incorporated by reference herein.

### FIELD

The present disclosure relates to a hematopoietic stem cell (HSC) proliferation medium which includes a basal medium, thrombopoietin (TPO), stem cell factor (SCF), interleukin-3 (IL-3), interleukin-6 (IL-6), Flt-3 ligand (FL), insulin, transferrin, human serum albumin (HSA), pyrimido-indole derivative UM729, and aryl hydrocarbon receptor (AhR) antagonist StemRegenin 1 (SR1). The disclosure also relates to methods for in vitro proliferation of hematopoietic stem cells (HSCs) and in vitro production of hematopoietic progenitor cells (HPCs) using the HSC proliferation medium.

### BACKGROUND

Hematopoietic stem cells (HSCs) refer to a group of stem cells which are capable of self-renewal and expressing CD 34 antigens, which can differentiate into various types of blood cells, and which are located in bone marrow, peripheral blood, and umbilical cord blood.

During blood cell development, HSCs differentiate into various hematopoietic progenitor cells (HPCs), including colony-forming unit-granulocyte/erythroid/macrophage/megakaryocyte (CFU-GEMM) colonies, burst-forming unit-erythroid (BFU-E) colonies, colony-forming unit-erythroid (CFU-E) colonies, and colony-forming unit-granulocyte/macrophage (CFU-GM) colonies. In particular, the CFU-GEMM colonies can further differentiate into the BFU-E, CFU-E, and CFU-GM colonies. Subsequently, the BFU-E and the CFU-E colonies will differentiate into erythrocytes, while the CFU-GM colonies will differentiate into granulocytes and macrophages.

HSCs have been clinically applied to reconstitute and restore the hematopoietic system for treating various hematologic disorders based on differentiation potential of these cells. However, sustaining HSCs in their stem cell state while enabling large-scale proliferation for downstream applications remains a substantial challenge, posing a bottleneck in hematological and immunological therapies.

In view of the aforesaid, there is still a need to develop an effective way for proliferating HSCs and for production of HPCs from the HSCs.

### SUMMARY

Therefore, in a first aspect, the present disclosure provides a hematopoietic stem cell (HSC) proliferation medium, which can alleviate at least one of the drawbacks of the prior art. The HSC proliferation medium includes a basal medium, 0.1 ng/mL to 200.0 ng/mL of thrombopoietin (TPO), 0.1 ng/mL to 200.0 ng/mL of stem cell factor (SCF), 0.1 ng/mL to 20.0 ng/mL of interleukin-3 (IL-3), 0.1 ng/mL to 100.0 ng/mL of interleukin-6 (IL-6), 0.1 ng/mL to 200.0 ng/mL of Flt-3 ligand (FL), 0.1 μg/mL to 20.0 μg/mL of insulin, 0.1 μg/mL to 120.0 μg/mL of transferrin, 0.1 g/L to 3.0 g/L of human serum albumin (HSA), 0.1 μM to 4.0 μM of pyrimido-indole derivative UM729, and 0.1 μM to 2.0 μM of aryl hydrocarbon receptor (AhR) antagonist StemRegenin 1 (SR1).

In a second aspect, the present disclosure provides a method for in vitro proliferation of hematopoietic stem cells (HSCs), which can alleviate at least one of the drawbacks of the prior art, and which includes cultivating a population of the HSCs in the aforesaid HSC proliferation medium.

In a third aspect, the present disclosure provides a method for in vitro production of hematopoietic progenitor cells (HPCs), which can alleviate at least one of the drawbacks of the prior art, and which includes: cultivating a population of HSCs in the aforesaid HSC proliferation medium, so as to obtain proliferated HSCs; and cultivating the proliferated HSCs in a differentiation medium, so as to obtain the HPCs derived from the proliferated HSCs. The HSCs are selected from the group consisting of induced pluripotent stem cell-derived hematopoietic stem cells (iPSC-HSCs) and umbilical cord blood-derived hematopoietic stem cells (CB-HSCs). When the HSCs are the iPSC-HSCs, the HPCs thus obtained are colony-forming unit-granulocyte/macrophage (CFU-GM) colonies. When the HSCs are the CB-HSCs, the HPCs thus obtained are burst-forming unit-erythroid (BFU-E) colonies and colony-forming unit-erythroid (CFU-E) colonies.

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a hematopoietic stem cell (HSC) proliferation medium, which includes a basal medium, 0.1 ng/mL to 200.0 ng/mL of thrombopoietin (TPO), 0.1 ng/mL to 200.0 ng/mL of stem cell factor (SCF), 0.1 ng/mL to 20.0 ng/mL of interleukin-3 (IL-3), 0.1 ng/mL to 100.0 ng/mL of interleukin-6 (IL-6), 0.1 ng/mL to 200.0 ng/mL of Flt-3 ligand (FL), 0.1 μg/mL to 20.0 μg/mL of insulin, 0.1 μg/mL to 120.0 μg/mL of transferrin, 0.1 g/L to 3.0 g/L of human serum albumin (HSA), 0.1 μM to 4.0 μM of pyrimido-indole derivative UM729, and 0.1 μM to 2.0 μM of aryl hydrocarbon receptor (AhR) antagonist StemRegenin 1 (SR1).

As used herein, the term “basal medium” refers to any culture medium that provides essential nutrients, such as a carbon source and a nitrogen source, to support in vitro growth of hematopoietic stem cells (HSCs).

According to the present disclosure, the basal medium suitable for cultivation of the HSCs may be prepared using techniques well-known to those skilled in the art, or may be obtained as a commercial product. In certain embodiments, the basal medium is selected from the group consisting of an Iscove's modified Dulbecco's medium (IMDM), a Dulbecco's modified Eagle's medium (DMEM), a Roswell Park Memorial Institute (RPMI) 1640 medium, a minimum essential medium (MEM), a basal medium Eagle (BME), an F-12K nutrient mixture medium, and combinations thereof. In an exemplary embodiment, the basal medium is the IMDM.

In certain embodiments, the HSC proliferation medium includes the IMDM serving as the basal medium, 100.0 ng/mL of the TPO, 100.0 ng/mL of the SCF, 10.0 ng/mL of the IL-3, 50.0 ng/mL of the IL-6, 100.0 ng/mL of the FL, 10.0 μg/mL of the insulin, 60.0 μg/mL of the transferrin, 1.5 g/L of the HSA, 2.0 μM of the UM729, and 1.0 μM of the SR1.

The present disclosure also provides a method for in vitro proliferation of HSCs, which includes cultivating a population of the HSCs in the aforesaid HSC proliferation medium.

As used herein, the term “hematopoietic stem cells (HSCs)” is intended to encompass the HSCs that are readily accessible to those skilled in the art (e.g., commercially available HSCs) or that are produced using techniques well-known to those skilled in the art.

In certain embodiments, the HSCs are selected from the group consisting of induced pluripotent stem cell-derived hematopoietic stem cells (iPSC-HSCs) and umbilical cord blood-derived hematopoietic stem cells (CB-HSCs).

According to the present disclosure, the iPSC-HSCs may be produced by subjecting human-derived induced pluripotent stem cells (iPSCs) to a differentiation process. The iPSCs may be obtained as a commercial product, or may be prepared using techniques well-known to those skilled in the art. In an exemplary embodiment, the iPSCs are prepared by subjecting human peripheral blood mononuclear cells to a reprogramming process with a Sendai virus vector carrying Oct4 (Octamer-binding transcription factor 4), Sox2 (sex determining region Y-box 2), Klf4 (Krüppel-like factor 4), and c-Myc (MYC proto-oncogene) genes.

According to the present disclosure, the CB-HSCs may be isolated from human fetal umbilical cord blood.

As used herein, the term “cultivating” can be used interchangeably with other terms such as “cultivation.” The procedures and conditions for the cultivation of the HSCs are within the expertise and routine skills of those skilled in the art.

According to the present disclosure, the cultivation of the HSCs in the HSC proliferation medium may be conducted at a temperature ranging from 36° C. to 37° C. for a time period ranging from 7 days to 14 days. In certain embodiments, the cultivation of the HSCs in the HSC proliferation medium is conducted at a temperature of 37° C. for a time period of 7 days.

The present disclosure also provides a method for in vitro production of hematopoietic progenitor cells (HPCs), which includes: cultivating a population of HSCs in the aforesaid HSC proliferation medium, so as to obtain proliferated HSCs; and cultivating the proliferated HSCs in a differentiation medium, so as to obtain the HPCs derived from the proliferated HSCs. The HSCs are selected from the group consisting of the iPSC-HSCs and the CB-HSCs. When the HSCs are the iPSC-HSCs, the HPCs thus obtained are colony-forming unit-granulocyte/macrophage (CFU-GM) colonies. When the HSCs are the CB-HSCs, the HPCs thus obtained are burst-forming unit-erythroid (BFU-E) colonies and colony-forming unit-erythroid (CFU-E) colonies.

As used herein, the term “differentiation medium” refers to any culture medium that is capable of inducing differentiation of HSCs.

According to the present disclosure, the differentiation medium suitable for inducing HSCs differentiation may be prepared using techniques well-known to those skilled in the art, or may be obtained as a commercial product. The differentiation medium may be adjusted based on a source or type of the HSCs used and the expertise of those skilled in the art.

In certain embodiments, the differentiation medium suitable for inducing iPSC-HSCs differentiation is a MethoCult™ SF H4636 medium (Manufacturer: StemCell Technologies, Cat. no.: 04636).

In certain embodiments, the differentiation medium suitable for inducing CB-HSCs differentiation is a MethoCult™ H4434 Classic medium (Manufacturer: StemCell Technologies, Cat. no.: 04434).

According to the present disclosure, the procedures and conditions for cultivating the proliferated HSCs with the differentiation medium may be adjusted according to practical requirements. In this regard, those skilled in the art may refer to operational guidelines, e.g., operational guidelines of the commercially available differentiation media mentioned above.

According to the present disclosure, cultivation of the proliferated HSCs in the differentiation medium may be conducted at a temperature ranging from 36° C. to 37° C. for a time period ranging from 7 days to 14 days. In certain embodiments, the cultivation of the proliferated HSCs in the differentiation medium is conducted at a temperature of 37° C. for a time period of 12 days.

The disclosure will be further described by way of the following examples. However, it should be understood that the following examples are solely intended for the purpose of illustration and should not be construed as limiting the disclosure in practice.

### EXAMPLES

### General Experimental Materials

1. Recombinant human cytokines and chemicals:

The types and relevant information regarding recombinant human cytokines and chemicals used to prepare a hematopoietic stem cell (HSC) proliferation medium of the present disclosure are shown in Table 1 below.

TABLE 1

| Type | Manufacturer | Cat. no. |
|---|---|---|
| Thrombopoietin (TPO) | PeproTech | AF-300-18 |
| Stem cell factor (SCF) | PeproTech | AF-300-07 |
| Interleukin-3 (IL-3) | PeproTech | AF-200-03 |
| Interleukin-6 (IL-6) | PeproTech | 200-06 |
| Flt-3 ligand (FL) | PeproTech | 300-19 |
| Transferrin | Sigma-Aldrich | T0665 |
| Insulin | Gibco | 12585014 |
| Human serum albumin (HSA) | Akron Biotech | AK8228- |
| | | 0100 |
| Pyrimido-indole derivative UM729 | StemCell Technologies | 72332 |
| Aryl hydrocarbon receptor (AhR) | Selleck Chemicals | S2858 |
| antagonist StemRegenin 1 (SR1) | LLC | |

2. An Iscove's modified Dulbecco's medium (IMDM, Cat. no.: SH30228.02) serving as a basal medium in the following experiments was purchased from Hyclone Laboratories, Inc.
3. Commercially available media:

The relevant information regarding three commercially available HSC proliferation medium (hereafter abbreviated as commercially available media 1 to 3) used for comparison is listed in Table 2 below. To be specific, the commercially available medium 3 was prepared by adding StemSpan™ CD34+ Expansion Supplement into StemSpan™-XF.

TABLE 2

| Commercially | | | |
|---|---|---|---|
| available | | | |
| medium | Trade name | Manufacturer | Cat. no. |
| 1 | HSC-Brew GMP | Miltenyi Biotec | 170-076-310 |
| | Medium | | |
| 2 | CellGenix ® GMP Stem | CellGenix | 20802-0500 |
| | Cell Growth Medium | | |
| | (SCGM), serum-free, | | |
| | xeno-free | | |
| 3 | StemSpan ™-XF | StemCell | 100-0073 |
| | StemSpan ™ CD34+ | Technologies | 02691 |
| | Expansion Supplement | | |

4. Induced pluripotent stem cell-derived hematopoietic stem cells (iPSC-HSCs) used in the following experiments were prepared by subjecting human peripheral blood mononuclear cell-derived induced pluripotent stem cells (PBMC-iPSCs) to a differentiation process using a STEMdiff™ Hematopoietic Kit (Manufacturer: StemCell Technologies., Cat. no.: 05310) in accordance with the manufacturer's instructions.
5. Umbilical cord blood-derived hematopoietic stem cells (CB-HSCs) used in the following experiments were isolated from human fetal umbilical cord blood with reference to the method described in U.S. Pat. No. 8,762,074 B2.

### Example 1. Preparation of Hematopoietic Stem Cell (HSC) Proliferation Medium of the Present Disclosure

The HSC proliferation medium of the present disclosure was prepared using the recipe shown in Table 3 below.

TABLE 3

| Ingredient | Concentration | |
|---|---|---|
| TPO | 100.0 | ng/mL |
| SCF | 100.0 | ng/mL |
| IL-3 | 10.0 | ng/mL |
| IL-6 | 50.0 | ng/mL |
| FL | 100.0 | ng/mL |
| Insulin | 10.0 | μg/mL |
| Transferrin | 60.0 | μg/mL |
| HSA | 1.5 | g/L |
| UM729 | 2.0 | μM |
| SR1 | 1.0 | μM |

Formulated in IMDM medium

### Example 2. Evaluation of the Effect of HSC Proliferation Medium of the Present Disclosure on In Vitro Proliferation of CD34⁺ HSCs

### A. Analysis of Ability of HSCs to Proliferate In Vitro

### Experimental Procedures:

First, the iPSC-HSCs prepared in section 4 of “General Experimental Materials” above were divided into 4 groups, including an experimental group and three comparative groups (i.e., comparative groups 1 to 3). Each group of the iPSC-HSCs was seeded at an initial count of 3×10⁵ cells in a T-25 flask containing 3 mL of a respective one of HSC proliferation media as shown in Table 4 below.

TABLE 4

| Group | HSC proliferation medium |
|---|---|
| Experimental group | HSC proliferation medium of the present |
| | disclosure |
| Comparative group 1 | Commercially available medium 1 |
| Comparative group 2 | Commercially available medium 2 |
| Comparative group 3 | Commercially available medium 3 |

Subsequently, each group of the iPSC-HSCs was cultured in an incubator (37° C., 5% CO₂) for 7 days, followed by determining the iPSC-HSCs count using a total nucleated cell (TNC) count.

### Results:

TABLE 5

| Group | iPSC-HSCs count (×10⁴, cells) |
|---|---|
| Experimental group | 228.7 ± 20.4** |
| Comparative group 1 | 25.4 ± 1.9 |
| Comparative group 2 | 38.7 ± 5.7 |
| Comparative group 3 | 95.7 ± 8.8 |

Note:
The symbol “**” represents p < 0.01 (compared with the comparative groups 1 to 3).

Table 5 shows the iPSC-HSCs count determined in each group of the iPSC-HSCs after 7 days of cultivation in the respective one of the HSC proliferation media. As shown in Table 5, compared with the initial count (i.e., 3×10⁵ cells), the iPSC-HSCs count determined in the experimental group showed a significant increase, i.e., nearly 8-fold increase, after being cultured with the HSC proliferation medium of the present disclosure. In contrast, the iPSC-HSCs count determined in each of the comparative groups 1 to 3 showed no significant increase after being cultured with a respective one of the corresponding commercially available media 1 to 3, with only the comparative group 3 achieving a 3-fold increase. In particular, the iPSC-HSCs of the comparative group 1 did not even proliferate after being cultured with the commercially available medium 1. These results demonstrate that the HSC proliferation medium of the present disclosure can effectively enhance in vitro proliferation of HSCs, and such effect is significantly better than that of other commercially available media.

### B. Determination of CD34⁺ Cell Percentage

### Experimental Procedures:

Based on the results described in section A of this example, cell cultures of the experimental group and the comparative group 3 were subjected to determination of CD34⁺ cell percentage (%).

First, a respective one of the cell cultures of the experimental group and the comparative group 3 was subjected to a centrifugation treatment at 1,000 rpm for 5 minutes, followed by removing the resultant cell culture supernatant, so as to collect the resultant cell pellet. The cell pellet was then washed with Dulbecco's phosphate buffered saline (DPBS) two times, followed by adding an appropriate amount of DPBS to suspend the washed cell pellet, so as to obtain a cell suspension. Subsequently, the cell suspension was added with a phycoerythrin (PE)-conjugated CD34 antibody solution (Manufacturer: BD Biosciences, Cat. no.: 340669, diluted 6-fold with DPBS), followed by incubating in the dark at 4° C. for 30 minutes, so as to obtain an incubated cell suspension. Thereafter, 2 mL of DPBS was added in the incubated cell suspension, followed by conducting a centrifugation treatment at 2,000 rpm for 5 minutes, and then the resultant supernatant was removed, so as to collect the resultant pellet. Afterwards, the pellet was washed with DPBS, followed by adding an appropriate amount of DPBS to suspend the washed pellet, so as to obtain a test sample. Thereafter, the test sample was subjected to determination of CD34⁺ cell count using a flow cytometer (Manufacturer: BD Biosciences, Model no.: FACSCanto II), where cells in the test sample were excited by an argon-ion laser with a wavelength of 488 nm, and fluorescence intensity of PE was then detected at a wavelength of 585 nm, with 10,000 cells analyzed each time. The thus obtained data were analyzed using BD FACSDiva™ Software, so as to determine the CD34⁺ cell percentage (%) in each group.

### Results:

The experimental results showed that the CD34⁺ cell percentage determined in the experimental group was 83.5%±2.6%, which was substantially higher than 40.6%±5.5% determined in the comparative group 3. These results indicate that the HSC proliferation medium of the present disclosure can effectively enhance in vitro proliferation of HSCs, with a differentiation capacity of the thus proliferated HSCs substantially outperforming that of HSCs cultured with the other commercially available media.

### Example 3. Evaluation of the Effect of HSC Proliferation Medium of the Present Disclosure on Ability of HSCs to Differentiate into Various Hematopoietic Progenitor Cells (HPCs) In Vitro

### Experimental Procedures:

A. Analysis of Ability of Proliferated iPSC-HSCs to Differentiate into HPCs

First, the iPSC-HSCs prepared in section 4 of “General Experimental Materials” above were divided into 2 groups, including a control group and an experimental group. The iPSC-HSCs of the experimental group were seeded at an initial count of 3×10⁵ cells in a T-25 flask containing 3 mL of the HSC proliferation medium of the present disclosure, followed by cultivation in an incubator (37° C., 5% CO₂) for 7 days, so as to obtain the proliferated iPSC-HSCs to be used in the following experiments. In addition, the iPSC-HSCs of the control group were obtained directly from the iPSC-HSCs (i.e., non-proliferated iPSC-HSCs) prepared in section 4 of “General Experimental Materials” above, without being cultured in any proliferation medium.

Next, each group of the iPSC-HSCs was seeded at an initial count of 3×10³ cells in a 3.5-cm Petri dish containing 1 mL of MethoCult™ SF H4636 medium (serving as a differentiation medium, Manufacturer: StemCell Technologies, Cat. no.: 04636), followed by cultivation in an incubator (37° C., 5% CO₂) for a time period ranging from 7 days to 14 days until cell colonies were formed. Thereafter, each group of the cell colonies formed in the 3.5-cm Petri dish was observed and photographed using an inverted microscope (Manufacturer: ZEISS, Model no.: Primovert) at a magnification of 100×, followed by analyzing size and morphology of the cell colonies, so as to determine percentages of HPCs at different differentiation stages among the cell colonies. The HPCs at different differentiation stages included burst-forming unit-erythroid (BFU-E) colonies, colony-forming unit-erythroid (CFU-E) colonies, colony-forming unit-granulocyte/macrophage (CFU-GM) colonies, and colony-forming unit-granulocyte/erythroid/macrophage/megakaryocyte (CFU-GEMM) colonies.

B. Analysis of Ability of Proliferated CB-HSCs to Differentiate into HPCs

First, the CB-HSCs prepared in section 5 of “General Experimental Materials” above were divided into 2 groups, including a control group and an experimental group. The CB-HSCs of the experimental group were seeded at an initial count of 3×10⁵ cells in a T-25 flask containing 3 mL of the HSC proliferation medium of the present disclosure, followed by cultivation in an incubator (37° C., 5% CO₂) for 7 days, so as to obtain the proliferated CB-HSCs to be used in the following experiments. In addition, the CB-HSCs of the control group were obtained directly from the CB-HSCs (i.e., non-proliferated CB-HSCs) prepared in section 5 of “General Experimental Materials” above, without being cultured in any proliferation medium.

Next, each group of the CB-HSCs was seeded at an initial count of 1×10² cells in a 3.5-cm Petri dish containing 1 mL of MethoCult™ H4434 Classic medium (serving as a differentiation medium, Manufacturer: StemCell Technologies, Cat. no.: 04434), followed by cultivation in an incubator (37° C., 5% CO₂) for a time period ranging from 7 days to 14 days until cell colonies were formed. Thereafter, each group of the cell colonies formed in the 3.5-cm Petri dish was observed and photographed using the inverted microscope at a magnification of 100×, followed by analyzing size and morphology of the cell colonies, so as to determine percentages of HPCs at different differentiation stages among the cell colonies. The HPCs at different differentiation stages included BFU-E colonies, CFU-E colonies, CFU-GM colonies, and CFU-GEMM colonies.

### Results:

TABLE 6

| | | | Experimental |
|---|---|---|---|
| HSCs | HPCs | Control group | group |
| iPSC-HSCs | BFU-E and CFU-E | 17.8%^{a} | 0%^{a} |
| | colonies | | |
| | CFU-GM colonies | 77.0% | 100% |
| | CFU-GEMM colonies | 5.2% | 0% |
| CB-HSCs | BFU-E and CFU-E | 26.5%^{a} | 58.8%^{a} |
| | colonies | | |
| | CFU-GM colonies | 41.2% | 29.4% |
| | CFU-GEMM colonies | 32.3% | 11.8% |

Note:
The symbol “^{a}” represents the sum of percentages of the BFU-E and CFU-E colonies.

Table 6 shows the percentage of the HSCs (i.e., the iPSC-HSCs and CB-HSCs) in each group that is differentiated into various HPCs at different differentiation stages. As shown in Table 6, with regard to the iPSC-HSCs, the HPCs observed in the control group included the BFU-E, CFU-E, CFU-GM, and CFU-GEMM colonies, whereas the HPCs observed in the experimental group were exclusively the CFU-GM colonies. In particular, the percentage of the CFU-GM determined in experimental group was 100%, which was much higher than 77.0% determined in the control group. These results demonstrate that the iPSC-HSCs cultivated in the HSC proliferation medium of the present disclosure can be subjected to proliferation, and the thus proliferated iPSC-HSCs can be directed to differentiate into immune cells (e.g., granulocytes and macrophages).

In addition, as for the CB-HSCs, the HPCs observed in the control group included the BFU-E, CFU-E, CFU-GM, and CFU-GEMM colonies, with the CFU-GM colonies being the most predominant colonies. Although the HPCs observed in the experimental group also included the aforesaid four colonies, the BFU-E and CFU-E colonies are the most predominant colonies. In particular, the sum of the percentages of the BFU-E and CFU-E colonies determined in the experimental group were more than twice higher than that in the control group. These results indicate that the CB-HSCs cultivated in the HSC proliferation medium of the present disclosure can be subjected to proliferation, and the thus proliferated CB-HSCs can be directed to differentiate into erythroid cells.

Summarizing the above test results, it is clear that the HSC proliferation medium of the present disclosure can effectively enhance in vitro proliferation of the HSCs from different source (e.g., the iPSC-HSCs and CB-HSCs), and the thus proliferated HSCs can be directed to differentiate into specific types of cells (such as immune cells or erythroid cells).

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, the one or more features may be singled out and practiced alone without the another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A hematopoietic stem cell (HSC) proliferation medium, comprising:
a basal medium;
0.1 ng/mL to 200.0 ng/mL of thrombopoietin (TPO);
0.1 ng/mL to 200.0 ng/mL of stem cell factor (SCF);
0.1 ng/mL to 20.0 ng/mL of interleukin-3 (IL-3);
0.1 ng/mL to 100.0 ng/mL of interleukin-6 (IL-6);
0.1 ng/mL to 200.0 ng/mL of Flt-3 ligand (FL);
0.1 μg/mL to 20.0 μg/mL of insulin;
0.1 μg/mL to 120.0 μg/mL of transferrin;
0.1 g/L to 3.0 g/L of human serum albumin (HSA);
0.1 μM to 4.0 μM of pyrimido-indole derivative UM729; and
0.1 μM to 2.0 μM of aryl hydrocarbon receptor (AhR) antagonist StemRegenin 1 (SR1).

2. The HSC proliferation medium as claimed in claim 1, wherein the basal medium is selected from the group consisting of an Iscove's modified Dulbecco's medium (IMDM), a Dulbecco's modified Eagle's medium (DMEM), a Roswell Park Memorial Institute (RPMI) 1640 medium, a minimum essential medium (MEM), a basal medium Eagle (BME), an F-12K nutrient mixture medium, and combinations thereof.

3. The HSC proliferation medium as claimed in claim 2, including:
the IMDM serving as the basal medium;
100.0 ng/mL of the TPO;
100.0 ng/mL of the SCF;
10.0 ng/mL of the IL-3;
50.0 ng/mL of the IL-6;
100.0 ng/mL of the FL;
10.0 μg/mL of the insulin;
60.0 μg/mL of the transferrin;
1.5 g/L of the HSA;
2.0 μM of the pyrimido-indole derivative UM729; and
1.0 μM of the AhR antagonist SR1.

4. A method for in vitro proliferation of hematopoietic stem cells (HSCs), comprising cultivating a population of the HSCs in the HSC proliferation medium as claimed in claim 1.

5. The method as claimed in claim 4, wherein the HSCs are selected from the group consisting of induced pluripotent stem cell-derived hematopoietic stem cells (iPSC-HSCs) and umbilical cord blood-derived hematopoietic stem cells (CB-HSCs).

6. A method for in vitro production of hematopoietic progenitor cells (HPCs), comprising:
cultivating a population of HSCs in the HSC proliferation medium as claimed in claim 1, so as to obtain proliferated HSCs, the HSCs being selected from the group consisting of iPSC-HSCs and CB-HSCs; and
cultivating the proliferated HSCs in a differentiation medium, so as to obtain the HPCs derived from the proliferated HSCs;
wherein,
when the HSCs are the iPSC-HSCs, the HPCs thus obtained are colony-forming unit-granulocyte/macrophage (CFU-GM) colonies,
when the HSCs are the CB-HSCs, the HPCs thus obtained are burst-forming unit-erythroid (BFU-E) colonies and colony-forming unit-erythroid (CFU-E) colonies.
